(19) United States
(12) Patent Application Publication
(10) Pub. No.: US 2025/0050006 A1
(43) Pub. Date: Feb. 13, 2025

(54) Heart deairing system

(71) Applicants: Shane Pullins, Minneapolis, MN (US); Daniel Benjamin Giddens, II, Monroe, GA (US)

(72) Inventors: Shane Pullins, Minneapolis, MN (US); Daniel Benjamin Giddens, II, Monroe, GA (US)

(73) Assignee: Profusion Services, LLC, Simpsonville, SC (US)

(21) Appl. No.: 18/777,470

(22) Filed: Jul. 18, 2024

Related U.S. Application Data
(60) Provisional application No. 63/527,577, filed on Jul. 18, 2023.

(51) Int. Cl. A61M 1/36; A61N 7/00; A61N 7/02

(52) U.S. Cl. CPC A61M 1/363; A61N 7/02; A61M 2205/103; A61M 2205/106; A61N 2007/0043

(57) ABSTRACT

A device and method for transmitting vibration to the heart during open-heart surgery in order to dislodge air bubbles formed along interior walls of the heart so that they may be removed from the body, rather than allowed to enter the circulatory system.

This non-provisional application claims the benefit of provisional application No. 63/527,577 filed Jul. 18, 2023.

### FIELD OF THE INVENTION

The present invention generally relates to devices, systems, and methods for de-airing the heart during cardiac procedures, and it is specifically directed to a vibrating contact device adapted to be placed against a heart, during open-heart surgery, and to transmit vibration to the heart. The purpose of the invention is to dislodge from inner surfaces of the heart air bubbles that formed and adhered there while the heart was exposed to environmental air during open-heart surgery. This displacement enables the air bubbles to be captured by a vent, rather than escaping into the circulatory system, post-surgery, and creating a blockage in a blood vessel that may cause a serious health complication. Furthermore, the invention can be directed to such a device that employs a heart contact element which vibrates at a high enough frequency to cause fragmentation of air bubbles disposed within the heart during surgery and render them less likely to cause embolism if they happen to evade venting and enter the circulatory system.

### BACKGROUND OF THE INVENTION

In the field of cardiac surgery, it is well known that exposing the heart to environmental air in the way that occurs during such heart surgery—and especially open-heart surgery —presents a significant risk of air bubbles, or emboli, forming within chambers of the heart. Importantly, those formed air bubbles can be of such significant size that, if they enter the surgical patient's circulatory system, they have the potential to block blood vessels and induce heart attacks, strokes, and other serious health complications. Consequently, in the context of cardiac surgical procedures there is a recognized need to identify air bubbles that have formed along interior walls of the heart and, inasmuch possible, remove them from the entire body, rather than allow them to remain within the heart and, ultimately, escape to the circulatory system sometime after surgery.

To this end, customarily, a transesophageal echocardiogram (TEE) is performed to create an image of the heart which makes visible any unwanted air bubbles. Additionally, an intracardiac vent, formed by a needle and negative pressure suction, is temporarily placed within the aorta (typically) to capture air and expel it out of the heart to avert air bubbles from entering the head vessels or the systemic blood circulation. However, since some air bubbles deposited along interior walls of the heart might tend to remain adhered thereto and are not easily detached by suction force without some intervening act of manipulating the heart walls to dislodge those bubbles, techniques and tools to create that very dislodging effect have been developed in the prior art.

To wit, cardiac surgical personnel might be inclined to gently, directly massage the heart or jostle the patient's body in efforts to detach air bubbles from the inner walls of the heart and urge them into a suction vent. For another example, U.S. Pat. No. 10,842,631 to Karimov, et al. discloses a cardiac deairing system formed by an inflatable device coupled to a catheter which is to be inserted into the thoracic cavity and inserted adjacent to the heart. The inflatable device is activated to alternately inflate and deflate, thereby transmitting motion to the heart in a way that simulates a massage and deforms the shape of the heart to dislodge air bubbles deposited along its interior walls. Use of a catheter enables the inflatable device to be delivered to the heart during less invasive heart surgeries.

However, the very reason that a catheter would be used to place an inflatable device in contact with a heart is because, as a practical matter, a balloon-type contact device would be used to impart motion of a heart only during minimally invasive procedures in which the sternum remains closed and the balloon is introduced into the body through a small incision made in the chest wall. That is because the reciprocating inflation and deflation of the balloon would cause movement of a heart wall that the balloon is in contact with only provided that the balloon is sandwiched between that heart wall surface and either the pericardium, a lung, or the chest wall. In an open thoracic cavity environment in which an inflatable device in contact with the heart does is not also in contact with an opposing surface, the inflations and deflations of the contact device would not impart sufficient movement of the heart wall to dislodge air bubbles.

Furthermore, there is a tension between either moving a subject's body violently enough or applying impacts of sufficient magnitude to outer surfaces of a patient's heart to deform the heart sufficiently to break air bubbles free of their adherence to inner surfaces of the heart, on the one hand, and traumatizing the heart with those very movements or impacts on the other hand. Because of that dilemma, even properly performed manual massaging of the heart cannot be relied upon to be entirely effective in dislodging potentially dangerous air emboli within the heart so that they may be captured by intracardiac vents during surgery. Rather, it is well understood that some such air bubbles, invariably, will eventually exit a heart chamber by way of the circulatory system post-operation, rather than being expelled though a venting apparatus during surgery.

Consequently, the present inventors have developed an improved deairing system for use during open-heart surgery that can more safely and effectively dislodge air bubbles from their deposit sites along interior walls of the heart during cardiac surgery than do prior art techniques and devices. That system accomplishes this by utilizing a heart contact element that vibrates. In every embodiment, this vibration represents oscillation of the heart wall contact element at a substantially higher frequency than any counterpart inflatable contact element is practically cable of motioning, and in some embodiments, this vibratory oscillation occurs at an ultrasound frequency. In either case, relative to convention options, this reduces the effective deairing process time during surgery, and it reduces the overall volume of intracardiac air that is retained post-operation—which, in turn, minimizes the incidence of embolism-induced cardiac arrests and strokes that occur during operative and postoperative recovery periods. Additionally, within the scope of their invention, the present inventors recognize that high-frequency vibration of the heart contact element will not only dislodge, it will fragment such air bubbles into yet smaller-sized ones so that any intracardiac air that eludes expulsion through vents during surgery poses a significantly lower risk of embolism upon entering the circulatory system thereafter.

### SUMMARY OF THE INVENTION

It is an object of the present invention to provide a cardiac deairing device that is, relative to conventional such devices, more safe and more effective at dislodging air bubbles from interior walls of the heart—especially during open-heart surgery. In one aspect of the invention, the device features a paddle-like contact element to be placed in direct contact with the outer surface of a heart and is vibrated to impart vibration of that heart wall sufficient to dislodge air bubbles from their adherence to its inner surface. In another aspect of the invention, that contact element is operatively connected to a vibration source which transmits vibration to it. Vibration is mechanically conducted to the contact element from a vibration source, such as a vibration motor. Finally, in another aspect, the motor has a variable output speed to enable the frequency of the contact element's vibration to be selectively controlled.

It is another object of the present invention to provide a cardiac deairing system capable of transmitting high frequency vibration to the heart for the purpose of maximizing its air dislodging efficacy and minimizing intracardiac air bubble size. In another aspect of the invention, the heart contact element can be an ultrasound probe device that delivers a sound wave frequency of at least 20,000 Hz to the heart tissue-far greater than the vibration frequency that could realistically be delivered by prior art contact elements for mechanically deforming the heart (e.g., inflatable devices).

### BRIEF DESCRIPTION OF THE DRAWINGS

FIG. 1 is a diagrammatic view of a heart deairing device in accordance with the present invention being placed in contact with the outer wall of open-heart surgery patient's heart;

FIG. 2 is cross-sectional, front perspective view of a heart deairing device in accordance with the present invention; and

FIG. 3 is a diagrammatic view of a heart deairing device in accordance with the present invention, the view indicating the vibratory motion of the contact element of the device.

### DETAILED DESCRIPTION OF THE PREFERRED EMBODIMENTS

The present disclosure, as defined by the claims that follow, relates to a heart deairing device 1 that is principally formed by a contact paddle 10 that is fabricated and contoured to be placed in contact with the outer surface of the heart at various positions and is capable of transmit vibration waves to the heart surface. In a preferred embodiment depicted in the accompanying drawings, those vibration waves are generated by a mechanical vibration source and delivered to areas along a wall of the heart so that it vibrates and dislodges an air bubble(s) adhered to its inner surface. However, in another embodiment, the vibration waves are, in fact, sound waves generated by a transducer—and can even be at ultrasound frequency of 20,000 Hz or greater.

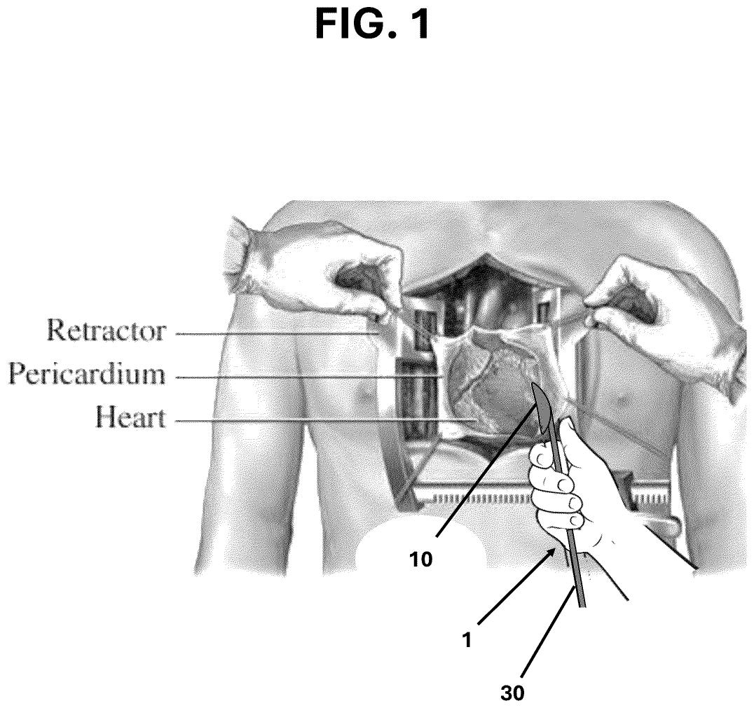

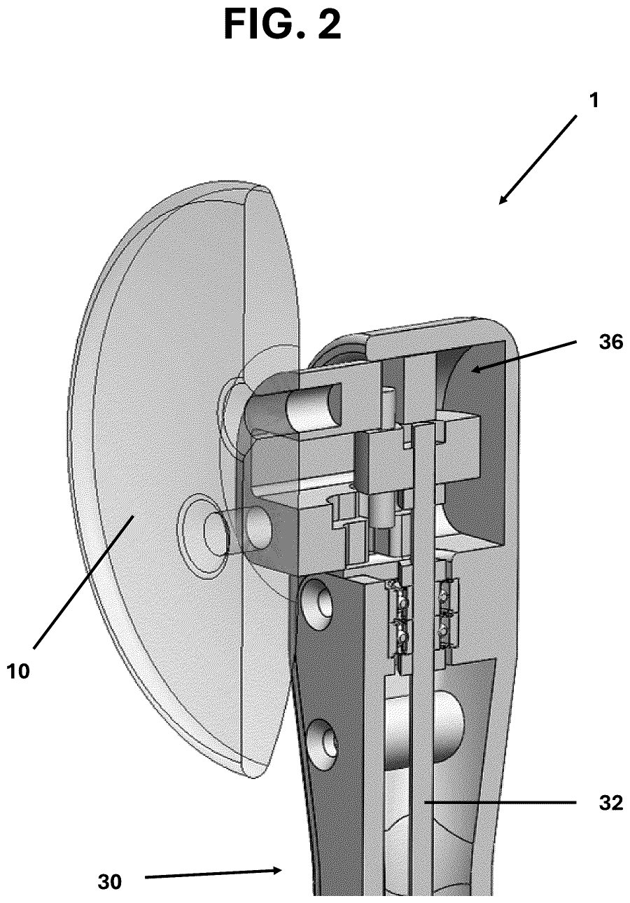

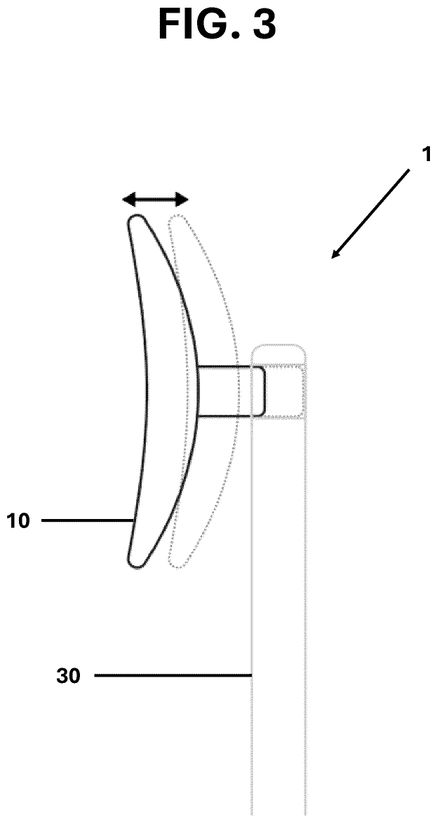

FIGS. 1-3 depict a version of the deairing device 1 of the present invention in which the heart contact paddle 10 is positioned at the proximal end of a handle 30. As can be gleaned from viewing FIG. 2, in this embodiment of the deairing device 1, the handle 30 is a tubular housing that encases a vibration source mechanism. In this shown example, the vibration source is formed by: (1) a motor (not shown) positioned toward the distal end of the handle 30; (2) a camshaft 32 that is axially rotated by and attached, at its distal end, to the motor; and (3) a cam-follower mechanism 36 that is operatively connected to both the camshaft 32 and the heart contact paddle 10 such that rotation of the camshaft 32 causes the cam-follower mechanism 36 to reciprocally push the paddle 10 away from the handle's axis and pull the paddle 10 back toward the handle's axis in such rapid succession that the paddle 10 vibrates. In a preferred embodiment, the operating speed of the motor can be adjusted so that paddle vibration frequency is selectively variable.

However, within the scope of the present invention, in another embodiment of the device not depicted in the accompanying drawings, the handle portion of the device either houses or connects the heart contact paddle 30 to an ultrasound transducer. In this embodiment, the transducer generates and can transmit to heart tissue, via the paddle 30, sound waves at 20,000 Hz plus frequencies.

It is understood that substitutions and equivalents for various elements set forth above may be obvious to those skilled in the art and may not represent a departure from the spirit of the invention. Therefore, the full scope and definition of the present invention is to be set forth by the claims that follow.

What is claimed is:

1. A heart deairing system comprising:
a heart contact element configured to place in contact with a heart and to transmit, to the heart, vibration at a frequency range sufficient to dislodge an air bubble from the heart's inner surface, wherein the contact element is non-inflatable; and
a vibration source for causing vibration of the contact element.

2. The heart deairing system of claim 1, wherein the vibration frequency of said contact element is controllably variable.

3. The heart deairing system of claim 1, wherein said vibration source is a vibration motor.

4. The heart deairing system of claim 1, wherein said vibration source comprises:
a camshaft;
a cam follower operatively connecting the camshaft to said contact element; and
a motor for rotating the camshaft, wherein camshaft rotation reciprocates the cam follower which, in turn, causes said contact element to vibrate.

5. The heart deairing system of claim 1, further comprising a handle that connects said contact element to said vibration source.

6. A heart deairing system comprising:
a heart contact element configured to place in contact with a heart and to conduct, to the heart, sound waves within a frequency range capable of fragmentizing and dislodging air bubbles from the heart's inner surface; and
a transducer for generating sound waves and conducting them to the heart contact element.

7. The heart deairing system of claim 6, wherein said transducer generates sound waves at a frequency of at least 20,000 Hz.

8. A method for deairing a heart, the method comprising:
identifying an air bubble that is disposed along an inner wall of the heart;
placing a non-inflatable contact device in contact with an outer wall of the heart proximate the identified air bubble;
placing a vent within the heart or aorta;
actuating the contact device to transmit vibration waves to the heart tissue and dislodge the air bubble from the heart inner wall, and
removing the dislodged air bubble from within the heart via the vent.

9. The method of claim 8, wherein the said contact device's vibration can be controlled and varied.

10. The method of claim 8, wherein said vibration waves are sound waves.

11. The method of claim 10, wherein said sound waves are at a frequency of at least 20,000 Hz and said contact device is an ultrasound probe.
